(19) United States
(12) Patent Application Publication
(10) Pub. No.: US 2026/0327599 A1
(43) Pub. Date: Oct. 22, 2026

(54) METHOD FOR SEPARATING LINEAR NUCLEIC ACID

(71) Applicant: KANEKA CORPORATION, Osaka (JP)

(72) Inventors: Shota Hirayama, Hyogo (JP); Teruyuki Nishi, Hyogo (JP); Koki Sasamoto, Hyogo (JP); Hirofumi Maeda, Hyogo (JP)

(73) Assignee: KANEKA CORPORATION, Osaka (JP)

(21) Appl. No.: 19/661,147

(22) Filed: Apr. 28, 2026

Related U.S. Application Data
(63) Continuation-in-part of application No. PCT/JP2025/021195, filed on Jun. 11, 2025.

(30) Foreign Application Priority Data
Aug. 20, 2024 (JP) 2024-139213

(51) Int. Cl. C12N 15/10

(52) U.S. Cl. CPC C12N 15/10

(57) ABSTRACT

A method for separating two or more linear nucleic acids having different molecular weights is provided. The method includes applying a liquid sample containing the two or more linear nucleic acids to a carrier having an immobilized anion exchange group to bind the two or more linear nucleic acids to the anion exchange group, applying an eluent of pH 4.0 to pH 6.5 to the carrier to elute an eluate containing a nucleic acid dissociated from the anion exchange group, and collecting the eluate. The applying the eluent includes increasing an electrical conductivity of the eluent in a continuous or stepwise manner based on the concentration change of a salt containing an alkali metal ion and/or an alkaline earth metal ion.

### TECHNICAL FIELD

One or more embodiments of the present invention relate to a method for separating linear nucleic acids, a method for producing a purified nucleic acid product, a kit for separating or purifying linear nucleic acids, and the like.

### BACKGROUND

A double-stranded circular DNA vector is widely used as an easily manipulable DNA vector. A double-stranded circular DNA vector comprises, in addition to a target gene sequence, an origin-of-replication sequence, an antibiotic resistance gene, and the like, which are sequences necessary for plasmid maintenance and replication. When a double-stranded circular DNA vector is introduced into an animal tissue or cell, these sequences are also delivered together with the target gene sequence, but such delivery is undesirable from the viewpoint of avoiding immunogenicity.

A linear covalently closed DNA (LCC DNA) is used as a means for removing unnecessary sequences from a double-stranded circular DNA vector and obtaining only the target gene sequence (Patent Literature 1).

A linear covalently closed DNA is a linear double-stranded DNA whose end portions are closed with a hairpin structure. A linear covalently closed DNA can be produced by utilizing the activity of a protelomerase to cleave a double-stranded DNA and form a hairpin structure at the cleaved ends. For example, a double-stranded DNA vector in which a target gene sequence is arranged between a pair of protelomerase recognition sequences is prepared. Next, by causing protelomerase activity to act on the double-stranded DNA vector, a linear covalently closed DNA comprising only the target gene sequence and having ends closed with a hairpin structure can be produced. The linear covalently closed DNA produced in this manner has resistance to an exonuclease, and thus is stable. The linear covalently closed DNA is also small in size, and thus, has an advantage of high efficiency of introduction into a cell. Additionally, by the arrangement of the protelomerase recognition sequences, a replication initiation region, an antibiotic resistance gene, and the like can be removed from the linear covalently closed DNA comprising the target gene sequence. Thus, a risk such as immunogenicity can be reduced.

In a reaction in which a linear covalently closed DNA is generated, in addition to the linear covalently closed DNA comprising a target gene, a linear covalently closed DNA that does not comprise the target gene but comprises sequences such as a replication initiation region and an antibiotic resistance gene is simultaneously generated as a byproduct. Patent Literature 2 discloses a method of degrading and removing the linear covalently closed DNA generated as a byproduct, using various enzymes. However, the method disclosed in Patent Literature 2 requires recombinant enzymes, and thus, causes a problem of high cost.

### PATENT LITERATURE

Patent Literature 1: U.S. Pat. No. 9,109,250B2
Patent Literature 2: WO2022/156188

### Non-Patent Literature

Non-Patent Literature 1: Sum, C. H., et al., Journal of Chromatography A, 1339 (2014) 214-218.

### SUMMARY

A method for efficiently separating a first linear covalently closed DNA comprising a target gene and a second linear covalently closed DNA generated as a byproduct in the production of the first linear covalently closed DNA, based on a difference in molecular weight, is provided.

As a method for binding and eluting a linear covalently closed DNA, a method using anion exchange column chromatography is known. The above-mentioned Non-Patent Literature 1 discloses a method of binding a plurality of linear covalently closed DNAs having two or more different molecular weights to an anion exchange membrane chromatograph, and then eluting the linear covalently closed DNAs using a basic buffer liquid having a pH of 8.1 as an eluent. However, the method has not achieved the separation of the linear covalently closed DNAs. In order to improve the collection rate of linear covalently closed DNAs, Non-Patent Literature 1 discloses that pH 9.0, which is a pH higher than pH 8.1, can be an ideal condition.

In order to address the above, the present inventors have attempted to separate each linear covalently closed DNA from a solution comprising two or more kinds of linear covalently closed DNAs having different molecular weights, using anion exchange column chromatography. The present inventors were not able to separate the linear covalently closed DNAs on the basis of the difference in molecular weight when a buffer liquid having a pH of 7.0 to 8.0 was used as an eluent. However, contrary to the above-mentioned Non-Patent Literature 1, the present inventors have found that linear covalently closed DNAs are efficiently separated when an eluent having a low pH of pH 4.0 to pH 6.5 is used.

Furthermore, the present inventors have evaluated various salts as a salt for increasing the electrical conductivity of the eluent having a pH of 4.0 to 6.5. As a result, the present inventors have found that linear nucleic acids can be efficiently separated using nine kinds of salts (sodium chloride, potassium chloride, potassium iodide, magnesium sulfate, calcium chloride, potassium bromide, sodium iodide, sodium bromide, and magnesium chloride). The present invention is based on the above findings and provides the following.

(1) A method for separating two or more linear nucleic acids having different molecular weights, comprising:
a binding step of applying a liquid sample containing the two or more linear nucleic acids to a carrier having an immobilized anion exchange group to bind the two or more linear nucleic acids to the anion exchange group;
an elution step of applying an eluent of pH 4.0 to pH 6.5 to the carrier to elute an eluate containing a nucleic acid dissociated from the anion exchange group; and
a collection step of collecting the eluate,
wherein the elution step comprises increasing the electrical conductivity of the eluent in a continuous or stepwise manner based on the concentration change of a salt containing an alkali metal ion and/or an alkaline earth metal ion.

(2) The method of (1), wherein the salt is a halide or a sulfate.

(3) The method of (2), wherein the halide is selected from the group consisting of sodium chloride, potassium chloride, potassium iodide, calcium chloride, potassium bromide, sodium iodide, sodium bromide, and magnesium chloride.

(4) The method of (2), wherein the sulfate is magnesium sulfate.

(5) The method of any one of (1) to (4), wherein the range for increasing the electrical conductivity encompasses at least part or all of the range of 0.1 mS/cm to 150 mS/cm.

(6) The method of any one of (1) to (5), wherein, in the collection step, the eluate eluted at different electrical conductivities are collected as different fractions.

(7) The method of (1), wherein the eluent comprises a buffer liquid selected from the group consisting of Bis-Tris buffer liquid, phosphate buffer liquid, histidine buffer liquid, citrate buffer liquid, acetate buffer liquid, and a buffer liquid in which any two or more thereof are combined.

(8) The method of (1), wherein the anion exchange group is a strong anion exchange group.

(9) The method of (8), wherein the strong anion exchange group comprises a quaternary amino group.

(10) The method of (9), wherein the quaternary amino group is a quaternary ammonium group, a quaternary aminoalkyl group, a quaternary aminoethyl group, and/or a trimethylaminoethyl group.

(11) The method of (1), wherein the carrier is a bead, a membrane, a gel, and/or a monolith.

(12) The method of (11), wherein the particle diameter of the bead is 1 μm to 10 μm.

(13) The method of (1), wherein the two or more linear nucleic acids are DNA.

(14) The method of (13), wherein the two or more linear nucleic acids are 2 kbp to 30 kbp.

(15) The method of (13) or (14), wherein the liquid sample further contains a circular DNA.

(16) The method of (1), wherein the two or more linear nucleic acids comprise a linear double-stranded DNA whose one or both ends are covalently closed ends.

(17) A method for producing a purified nucleic acid product, comprising:
a binding step of applying a liquid sample containing two or more linear nucleic acids having different molecular weights to a carrier having an immobilized anion exchange group to bind the two or more linear nucleic acids to the anion exchange group;
an elution step of applying an eluent of pH 4.0 to pH 6.5 to the carrier to elute a purified nucleic acid product dissociated from the anion exchange group; and
a collection step of collecting an eluate containing the purified nucleic acid product,
wherein the elution step comprises increasing the electrical conductivity of the eluent in a continuous or stepwise manner based on the concentration change of a salt containing an alkali metal ion and/or an alkaline earth metal ion, and
wherein the purified nucleic acid product contains all or part of the two or more linear nucleic acids in a ratio different from the ratio in the liquid sample.

(18) The method of (17), further comprising a preparation step of preparing a nucleic acid solution containing the purified nucleic acid product in a new buffer solution different from the eluate after the collection step.

(19) A kit for separating or purifying linear nucleic acids, comprising:
a carrier to which an anion exchange group is immobilized; and
an eluent of pH 4.0 to pH 6.5 for eluting nucleic acids, containing a salt containing an alkali metal ion and/or an alkaline earth metal ion.

(20) The kit of (19), wherein the electrical conductivity of the eluent for eluting nucleic acids is 0.1 mS/cm or higher.

(21) The kit of (19), wherein the anion exchange group is a strong anion exchange group.

(22) The kit of (21), wherein the strong anion exchange group comprises a quaternary amino group.

(23) The kit of (19), wherein the eluent for eluting nucleic acids comprises two or more eluents having different electrical conductivities.

(24) The kit of (19), wherein the carrier is a bead, a membrane, a gel, and/or a monolith.

(25) The kit of (24), wherein the bead has a particle diameter of 1 μm to 10 μm.

(26) The kit of (19), further comprising a wash liquid for washing the carrier to which the linear nucleic acid is bound.

Provided by one or more embodiments of the present invention is a method for efficiently separating a first linear covalently closed DNA comprising a target gene and a second linear covalently closed DNA generated as a byproduct in the production of the first linear covalently closed DNA, based on a difference in molecular weight.

### BRIEF DESCRIPTION OF DRAWINGS

FIGS. 1A-1C show the results of performing elution and fractionation under Experimental Conditions 1 to 3. The elution patterns of anion exchange column chromatography are shown in the upper parts of the panels, and the results of electrophoresis of the fractions eluted at the positions indicated by the arrows are shown in the lower parts of the panels. FIG. 1A shows the results of Experimental Condition 1. FIG. 1B shows the results of Experimental Condition 2. FIG. 1C shows the results of Experimental Condition 3.

FIGS. 2A-2C show the results of performing elution and fractionation under Experimental Conditions 4 to 6. The elution patterns of anion exchange column chromatography are shown in the upper parts of the panels, and the results of electrophoresis of the fractions eluted at the positions indicated by the arrows are shown in the lower parts of the panels. FIG. 2A shows the results of Experimental Condition 4. FIG. 2B shows the results of Experimental Condition 5. FIG. 2C shows the results of Experimental Condition 6.

FIGS. 3A and 3B show the results of performing elution and fractionation under Control Conditions 1 and 2. The elution patterns of anion exchange column chromatography are shown in the upper parts of the panels, and the results of electrophoresis of the fractions eluted at the positions indicated by the arrows are shown in the lower parts of the panels. FIG. 3A shows the results of Control Condition 1. FIG. 3B shows the results of Control Condition 2.

FIGS. 4A-4C show the results of performing elution and fractionation under Experimental Conditions (i) to (iii). The elution patterns of anion exchange column chromatography are shown on the upper panels, and the results of electrophoresis of the fractions eluted in the ranges indicated by the arrows are shown in the lower panels. FIG. 4A shows the results of Experimental Condition (i). FIG. 4B shows the results of Experimental Condition (ii). Note that two fraction groups indicated by one or two arrows correspond respectively between the elution pattern and the electrophoresis results. FIG. 4C shows the results of Experimental Condition (iii).

FIGS. 5A-5C show the results of performing elution and fractionation under experimental conditions (iv) to (vi). The elution patterns of anion exchange column chromatography are shown on the upper panels, and the results of electrophoresis of the fractions eluted in the ranges indicated by the arrows are shown in the lower panels. FIG. 5A shows the results of Experimental Condition (iv). FIG. 5B shows the results of Experimental Condition (v).

FIG. 5C shows the results of Experimental Condition (vi).

FIGS. 6A-6C show the results of performing elution and fractionation under experimental conditions (vii) to (ix). The elution patterns of anion exchange column chromatography are shown on the upper panels, and the results of electrophoresis of the fractions eluted in the ranges indicated by the arrows are shown in the lower panels. FIG. 6A shows the results of Experimental Condition (vii). FIG. 6B shows the results of Experimental Condition (viii). FIG. 6C shows the results of Experimental Condition (ix).

### DETAILED DESCRIPTION

### 1. Method for Separating Linear Nucleic Acid

### 1-1. Overview

One or more embodiments of the present invention are a method for separating two or more linear nucleic acids having different molecular weights (hereinafter referred to as a “method for separating linear nucleic acids” or simply “the separation method of one or more embodiments of the present invention”). The separation method of one or more embodiments of the present invention comprises a binding step, an elution step, and a collection step, and is characterized in that the elution of nucleic acids in the elution step dissociates the nucleic acids from an anion exchange group using an eluent having a pH of 4.0 to 6.5. Additionally, the elution step is characterized by increasing the electrical conductivity of the eluent in a continuous or stepwise manner based on the concentration change of a salt containing an alkali metal ion and/or an alkaline earth metal ion. According to the separation method of one or more embodiments, linear nucleic acids such as linear covalently closed DNA can be efficiently separated based on a difference in molecular weight.

### 1-2. Definitions of Terms

The following terms frequently used in the present specification are defined.

As used herein, the term “nucleic acid” is not particularly limited as long as the nucleic acid is two or more nucleotides linked by an internucleotide bond(s). For example, the nucleic acid may be either DNA or RNA. Additionally, the nucleic acid may be single-stranded, double-stranded, or more multi-stranded. The shape of the nucleic acid is not limited, and may be either linear or circular. The size of the nucleic acid is not limited as long as the size is 2 bases or more, and may be, for example, 8 bases or more in length, 10 bases or more in length, 20 bases or more in length, 50 bases or more in length, 100 bases or more in length, 200 bases or more in length, 500 bases or more in length, 1,000 bases or more in length, or 2,000 bases or more in length, and/or 100,000 bases or less in length, 50,000 bases or less in length, 20,000 bases or less in length, 15,000 bases or less in length, 10,000 bases or less in length, or 5,000 bases or less in length. Exemplary ranges include 10 bases to 100,000 bases in length, 100 bases to 60,000 bases in length, 500 bases to 50,000 bases in length, 1,000 bases to 40,000 bases in length, 2,000 bases to 30,000 bases in length, 3,000 bases to 20,000 bases in length, 4,000 bases to 10,000 bases in length, or 5,000 bases to 8,000 bases in length. Moreover, the size of a double-stranded nucleic acid may be 2 bp or more, and may be, for example, 8 bp or more, 10 bp or more, 20 bp or more, 50 bp or more, 100 bp or more, 200 bp or more, 500 bp or more, 1 kbp or more, or 2 kbp or more, and/or 100 kbp or less, 50 kbp or less, 20 kbp or less, 15 kbp or less, 10 kbp or less, or 5 kbp or less. Exemplary ranges include 10 bp to 100 kbp, 100 bp to 60 kbp, 500 bp to 50 kbp, 1 kbp to 40 kbp, 2 kbp to 30 kbp, 3 kbp to 20 kbp, 4 kbp to 10 kbp, or 5 kbp to 8 kbp. Examples of the molecular weight of the nucleic acid include a molecular weight corresponding to the above size. A method for calculating the molecular weight of a nucleic acid depending on the form such as DNA or RNA, or a single strand or a double strand, is well known in the art.

As used herein, the “linear nucleic acid” may be, for example, a linear DNA or a linear RNA. Additionally, the linear nucleic acid may be a linear single-stranded nucleic acid (for example, a linear single-stranded DNA) or a linear double-stranded nucleic acid (for example, a linear double-stranded DNA). Specific examples of the linear double-stranded DNA include a linear covalently closed DNA, a linear open-chain DNA, a restriction enzyme-cleaved DNA fragment, a PCR amplification product DNA, and the like. Specific examples of the linear single-stranded DNA include cDNA, a single-stranded DNA obtained by denaturing the above-mentioned linear double-stranded DNA, and the like.

As used herein, the “circular nucleic acid” may be, for example, a circular DNA. Additionally, the circular nucleic acid may be either a circular single-stranded nucleic acid (for example, a circular single-stranded DNA) or a circular double-stranded nucleic acid (for example, a circular double-stranded DNA). Specific examples of the circular double-stranded DNA include a plasmid DNA, a viral vector DNA, a viral genomic DNA, a bacmid vector, and the like. The shape of the circular double-stranded DNA is not particularly limited, and may be, for example, closed-circular or open-circular. The closed-circular nucleic acid may be supercoiled.

As used herein, the “linear covalently closed DNA (LCC DNA)” refers to a linear double-stranded DNA whose ends are closed with a hairpin structure. At each end of the linear covalently closed DNA, a 5′ end and a 3′ end of two DNA strands are ligated to form a hairpin structure. The linear covalently closed DNA has an end portion protected by a hairpin structure, and the end portion has resistance to degradation by exonucleases. The linear covalently closed DNA can be generated by a protelomerase recognizing a protelomerase recognition sequence, cleaving a double-stranded DNA molecule at a position within or near the protelomerase recognition sequence, and re-ligating the cleaved ends. Alternatively, the linear covalently closed DNA can be generated by using a DNA ligase to ligate a hairpin double-stranded DNA to one or both ends of a linear open-chain DNA cleaved with a restriction enzyme (restriction endonuclease) or the like (JP2023-126487A). Note that some linear covalently closed DNAs have one or both ends consisting of a covalently closed end(s). In some cases, a linear double-stranded DNA having only one end consisting of a covalently closed end is referred to as a “loop-shaped fragment”. However, in the present specification, the linear covalently closed DNA means a DNA in which both ends consist of covalently closed ends, unless otherwise specified.

As used herein, the “two or more linear nucleic acids” may be, for example, two, three, four, five, six, or more linear nucleic acids. The types and/or shapes of the two or more linear nucleic acids may be the same or different. For example, the two or more linear nucleic acids may be two or more linear double-stranded DNAs, for example, two or more linear covalently closed DNAs. Additionally, the two or more linear nucleic acids may be a combination of a linear covalently closed DNA and a linear double-stranded DNA whose ends are not closed with a hairpin structure.

The difference in size of the two or more linear nucleic acids separated by the separation method of the present invention is not particularly limited, and may be, for example, 8 bases or more in length, 10 bases or more in length, 20 bases or more in length, 50 bases or more in length, 100 bases or more in length, 200 bases or more in length, 500 bases or more in length, 1,000 bases or more in length, or 2,000 bases or more in length, and/or 20,000 bases or less in length, 15,000 bases or less in length, 10,000 bases or less in length, 5,000 bases or less in length, 4,000 bases or less in length, or 3,000 bases or less in length. Exemplary ranges include 10 bases to 10,000 bases in length, 100 bases to 8,000 bases in length, 200 bases to 6,000 bases in length, 500 bases to 5,000 bases in length, 1,000 bases to 4,000 bases in length, or 2,000 bases to 3,000 bases in length. Additionally, the difference in size of the two or more linear double-stranded nucleic acids may be, for example, 8 bp or more, 10 bp or more, 20 bp or more, 50 bp or more, 100 bp or more, 200 bp or more, 500 bp or more, 1 kbp or more, or 2 kbp or more, and/or 20 kbp or less, 15 kbp or less, 10 kbp or less, 5 kbp or less, 4 kbp or less, or 3 kbp or less. Exemplary ranges include 10 bp to 10 kbp, 100 bp to 8 kbp, 200 bp to 6 kbp, 500 bp to 5 kbp, 1 kbp to 4 kbp, or 2 kbp to 3 kbp. Examples of the difference in molecular weight of the two or more linear nucleic acids include a difference in molecular weight corresponding to the above difference in size.

As used herein, the “protelomerase” refers to a polypeptide having an activity to recognize a specific sequence in a double-stranded DNA molecule, cleave the double-stranded DNA molecule at a position within or near the recognition sequence, and ligate a 5′ end and a 3′ end at a cleaved end to form a covalently closed end (hereinafter referred to as “protelomerase activity”).

The base sequence of a linear nucleic acid such as a linear covalently closed DNA separated by the separation method of one or more embodiments of the present invention is not particularly limited, but may comprise a target nucleic acid sequence. As used herein, the “target nucleic acid sequence” is not limited to a sequence encoding a protein or a fragment thereof, or an RNA. The target nucleic acid sequence may be, for example, a sequence capable of functioning as a nucleic acid drug, and may be a sequence available for introducing the target nucleic acid sequence. Specific examples of the nucleic acid sequence include, but are not limited to: a gene sequence encoding a protein or a fragment thereof; a nucleic acid sequence encoding a non-coding RNA such as siRNA, shRNA, miRNA, lncRNA, or ribozyme; or a nucleic acid drug such as an antisense nucleic acid, an aptamer, or a bait nucleic acid. Additionally, examples of the sequence available for introducing the target nucleic acid sequence include a sequence recognizable/cleavable by a nuclease such as a restriction enzyme or a genome editing enzyme, a multicloning site comprising a sequence recognizable/cleavable by two or more nucleases, and the like. In one or more embodiments, the target nucleic acid sequence is a target gene sequence. As used herein, the “target gene sequence” refers to a gene sequence encoding a target protein or a fragment thereof, or a target RNA molecule. The target protein or a fragment thereof is not particularly limited and can be suitably selected according to the purpose. Examples include polypeptides constituting viruses, polypeptides produced by animals, plants, fungi, algae, bacteria, viruses, and the like, and fragments thereof. The polypeptides and fragments thereof can be used as a cell/gene therapeutic drug, a vaccine, a drug for treating a disease, and the like. The polypeptides produced by animals, plants, fungi, algae, bacteria, viruses, and the like are not particularly limited and can be suitably selected according to the purpose. Examples include: enzymes such as phytase, amylase, glucosidase, cellulase, lipase, protease, glutaminase, peptidase, oxidase, lactase, xylanase, trypsin, pectinase, and isomerase; antibody-binding proteins such as protein A, protein G, and protein L; antibodies or antibody fragments such as human antibodies, humanized antibodies, chimeric antibodies, llama antibodies, alpaca antibodies, single-chain antibodies, heavy-chain antibodies, multivalent antibodies, Fab, F(ab′), F(ab′)₂, Fc, Fc fusion proteins, bispecific antibodies, heavy chains (H chains), light chains (L chains), single-chain Fvs (scFvs), sc(Fv)₂, disulfide-linked Fvs (sdFvs), diabodies, and antibody-like molecule-targeting peptides (microantibodies); complexes (antibody-like molecules) of compounds other than antibodies or antibody fragments and antibody fragments, such as other functional proteins; serum albumins such as human serum albumin; epidermal growth factors such as human epidermal growth factor; insulin; growth hormone; erythropoietin; interferon; blood coagulation factor VIII; granulocyte colony-stimulating factor (G-CSF); granulocyte-macrophage colony-stimulating factor (GM-CSF); thrombopoietin; IL-1; IL-6; tissue plasminogen activator (TPA); urokinase; leptin; stem cell growth factor (SCF); fibroin; fluorescent protein; hepatitis B virus surface antigen; hirudin; and the like. Note that the target nucleic acid sequence such as the target gene sequence may also comprise a promoter in addition to the target nucleic acid sequence (for example, a sequence encoding a target protein or a fragment thereof). Additionally, the target nucleic acid sequence may further comprise a component(s) such as an intron, an enhancer, a terminator, and/or a poly(A) signal, if necessary.

As used herein, the “carrier” refers to a support to which an anion exchange group for binding a nucleic acid is immobilized. The material constituting the carrier is not limited, and may be, for example, agarose, sepharose, cellulose, glass, silica, polystyrene, collodion charcoal, polymethacrylate, zirconia, zeolite, vinyl polymer, polymethacrylate, polyacrylamide, polyacrylic acid, or dextran. The structure and shape of the carrier are not limited, and may be suitably selected from types such as a bead, a membrane, a gel, and/or a monolith. In any case, the carrier is washed, if necessary, after the nucleic acid is bound to the carrier, and the nucleic acid is eluted from the carrier and collected. The carrier is often packed into a container such as a column when used. For example, in column chromatography, a carrier such as a bead or a gel can be packed into a separation column when used. On the other hand, a carrier such as a magnetic bead does not necessarily need to be packed into a container such as a column, because such a carrier can be collected based on sedimentation, aggregation, precipitation, centrifugation, filtration, magnetic force, or the like.

As used herein, the “bead” refers to a particulate insoluble carrier. The average particle size or particle diameter of the bead may be, for example, 0.1 μm to 100 μm, 1 μm to 50 μm, 1 μm to 30 μm, or 2 μm to 20 μm. The material of the bead is not particularly limited, and is, for example, a polymer, a magnetic substance, silica, agarose, or sepharose. In particular, a bead consisting of a magnetic substance (magnetic bead) is easy to collect or purify based on a magnetic force.

As used herein, the “membrane” refers to a membrane carrier having pores for capturing a target molecule. The average pore size of the membrane is, for example, 0.1 to 100 μm, 0.2 to 10 μm, 0.3 to 2 μm, or 0.5 to 1 μm. The material of the membrane is not particularly limited, and is, for example, (hydrophilic) polyethersulfone, polyethylene, polypropylene, ethylene-vinyl acetate copolymer, polytetrafluoroethylene, polycarbonate, polyvinyl chloride, or polyamide. For example, by passing a liquid through the membrane placed in a container such as a column, a capsule, or a cartridge, a nucleic acid can be bound to the membrane, washed, and further eluted.

As used herein, the “gel” refers to a gel-like packing material. Usually, the gel is packed into a column and used for column chromatography. The material of the gel is not limited and may be either a styrene-based or agarose-based material. Examples of the material include sepharose, cellulose, agarose, and the like.

As used herein, the “monolith” refers to a porous ion exchange resin. The monolith is a porous carrier with a large pore size, and based on this, the monolith can purify large biomolecules quickly and gently. The average pore size of the monolith is, for example, 0.2 to 20 μm, 0.5 to 10 μm, 1 to 5 μm, or 2 to 3 μm. An example of a material of the monolith is a methacrylate-based polymer.

As used herein, the “anion exchange group” refers to a positively charged or positively chargeable group capable of binding an anion. The anion exchange group is also called an anion exchange ligand. An anion exchange group that maintains a constant charge state regardless of a pH change is called a strong anion exchange group, and an anion exchange group whose charge state changes depending on a pH change is called a weak anion exchange group. Specific examples of the anion exchange group include an amino group. The amino group may be a primary amino group, a secondary amino group, a tertiary amino group, and/or a quaternary amino group. A quaternary amino group is known as a strong anion exchange group. Specific examples of the quaternary amino group include a quaternary ammonium group, a quaternary aminoalkyl group, a quaternary aminoethyl group, and a trimethylaminoethyl group. Additionally, examples of the strong anion exchange group comprising a quaternary amino group include polyethyleneimine. Examples of the tertiary amino group include a diethylaminoethyl group and a diethylaminopropyl group, and such groups are classified as weak anion exchange groups.

As used herein, the term “separation” is not limited to completely separating a specific component from (an) other component(s), but the ratio of a target component may be increased compared to (an) other component(s). For example, “separating two or more linear nucleic acids” means that a content ratio of at least one of the two or more linear nucleic acids increases compared to the other linear nucleic acid(s). For example, obtaining a sample or a fraction in which the content ratio of a specific linear nucleic acid is increased compared to a sample before separation falls under this term.

As used herein, the term “elution” means that a liquid comprising a substance dissociated from a carrier (the liquid is referred to as an “eluate”) is separated from the carrier. A specific method of the elution is not limited, and may be, for example, elution based on gravity or pressure. Note that a liquid added to the carrier in order to elute the eluate is referred to as an “eluent”.

As used herein, the “liquid sample” is not limited as long as the sample comprises two or more linear nucleic acids. The liquid sample may be a sample comprising only two or more linear nucleic acids as nucleic acids, or may be a sample comprising a circular nucleic acid in addition to two or more linear nucleic acids.

### 1-3. Method

The separation method of one or more embodiments of the present invention comprises a binding step, an elution step, and a collection step as essential steps, and comprises an equilibration step, a washing step, and a purification step as optional steps. The constitution of each step will be described below.

### (Equilibration Step)

In the separation method of one or more embodiments of the present invention, the “equilibration step” is a step of equilibrating a carrier with a buffer liquid. This step is an optional step.

This step is performed by applying an equilibration buffer liquid to the carrier before the below-described binding step. The equilibration buffer liquid may be, for example, a buffer liquid having the same electrical conductivity and/or pH as a loading buffer liquid used in the below-described binding step. The amount of the equilibration buffer liquid used in this step may be 1 to 20 times the carrier volume, or 3 to 5 times the carrier volume. Additionally, the number of times the equilibration buffer liquid is added to the carrier is 1 or more, and may be, for example, 1, 2, 3, or 4. By this step, the carrier may be equilibrated to a pH and an electrical conductivity at which a nucleic acid can bind to an anion exchange group.

### (Binding Step)

In the separation method of the present invention, the “binding step” is a step of applying a liquid sample containing two or more linear nucleic acids to a carrier having an immobilized anion exchange group to bind the two or more linear nucleic acids to the anion exchange group. In this step, “applying” the liquid sample to the carrier can also be paraphrased as loading or applying. For example, loading the liquid sample into a container comprising the carrier to which the anion exchange group is immobilized (the container is, for example, a column packed with the carrier) corresponds to “applying”. In this step, the liquid sample is applied to the carrier, and the two or more linear nucleic acids in the liquid sample consequently bind to the anion exchange group.

The liquid sample to be applied to the carrier in the binding step may be prepared, before being applied to the carrier, to a pH and an electrical conductivity at which a nucleic acid can bind to the anion exchange group, by mixing the liquid sample with a loading buffer liquid or the like. The pH of the liquid sample applied to the carrier is, for example, 3 to 11, 4 to 10, 5 to 9, 6 to 8, or 6.5 to 7.5. Additionally, the electrical conductivity of the liquid sample applied to the carrier is, for example, 0.001 mS/cm to 150 mS/cm, 0.01 mS/cm to 100 mS/cm, 0.1 mS/cm to 50 mS/cm, 0.2 mS/cm to 10 mS/cm, 0.3 mS/cm to 5 mS/cm, or 0.5 mS/cm to 2 mS/cm. Note that in the present specification, “electrical conductivity” can be paraphrased as “ionic strength”. The pH and the electrical conductivity of the liquid sample can be adjusted by combining a buffer liquid for adjusting the pH (for example, Bis-Tris buffer liquid, phosphate buffer liquid, histidine buffer liquid, citrate buffer liquid, acetate buffer liquid, or a combination of any two or more thereof) and a salt for increasing the electrical conductivity (for example, sodium chloride, sodium iodide, sodium bromide, ammonium acetate, potassium chloride, potassium iodide, potassium bromide, magnesium chloride, magnesium sulfate, or calcium chloride).

### (Washing Step)

In the separation method of one or more embodiments of the present invention, the “washing step” is a step of washing the carrier after the binding step. This step is an optional step.

This step is a step of applying a washing buffer liquid to the carrier after the above-described binding step and before the below-described elution step to elute a component not bound to the anion exchange group, for example, a contaminant other than a nucleic acid, from the carrier. The washing buffer liquid used in this step is a solution used for removing a contaminant remaining on the carrier, and may be a solution that does not cause a nucleic acid to flow out of the carrier. For example, a buffer liquid having the same electrical conductivity and/or pH as the loading buffer liquid can be used. The amount of the washing buffer liquid used in the washing step may be 1 to 20 times the carrier volume packed with the carrier, or 3 to 5 times the carrier volume. Additionally, the number of times of washing is 1 or more, and may be, for example, 1, 2, 3, or 4.

### (Elution Step)

In the separation method of one or more embodiments of the present invention, the “elution step” is a step of applying an eluent of pH 4.0 to pH 6.5 to the carrier to elute an eluate comprising a nucleic acid dissociated from the anion exchange group.

The pH of the eluent used in this step is 4.0 to 6.5, and is, for example, 4.1 or higher, 4.2 or higher, 4.3 or higher, 4.4 or higher, 4.5 or higher, 4.6 or higher, 4.7 or higher, 4.8 or higher, 4.9 or higher, 5.0 or higher, 5.1 or higher, 5.2 or higher, 5.3 or higher, or 5.4 or higher, and/or 6.4 or lower, 6.3 or lower, 6.2 or lower, 6.1 or lower, 6.0 or lower, 5.9 or lower, 5.8 or lower, 5.7 or lower, or 5.6 or lower. Examples of a suitable range include 4.2 to 6.3, 4.5 to 6.0, 4.7 to 5.8, 5.0 to 5.5, or 5.2 to 5.4.

In this step, the eluent may be applied to the carrier while changing the electrical conductivity of the eluent in a continuous or stepwise manner. Here, the continuous or stepwise change is, in principle, a continuous or stepwise increase.

The range for changing the electrical conductivity encompasses at least part or all of a range of, for example, 0.1 mS/cm or higher, 0.5 mS/cm or higher, 1 mS/cm or higher, 2 mS/cm or higher, 5 mS/cm or higher, 10 mS/cm or higher, 20 mS/cm or higher, 30 mS/cm or higher, or 40 mS/cm or higher, and/or 150 mS/cm or lower, 140 mS/cm or lower, 130 mS/cm or lower, 120 mS/cm or lower, 110 mS/cm or lower, 100 mS/cm or lower, 90 mS/cm or lower, 80 mS/cm or lower, 70 mS/cm or lower, 60 mS/cm or lower, or 50 mS/cm or lower. In the range of 0.1 mS/cm to 150 mS/cm, a suitable range may be, for example, 1 mS/cm to 100 mS/cm, 5 mS/cm to 80 mS/cm, 10 mS/cm to 60 mS/cm, or 20 mS/cm to 50 mS/cm.

The eluent used in this step can be prepared, for example, by combining a salt for increasing the electrical conductivity and a buffer liquid for adjusting the pH (for example, Bis-Tris buffer liquid, phosphate buffer liquid, histidine buffer liquid, citrate buffer liquid, acetate buffer liquid, or a buffer liquid in which any two or more thereof are combined). In addition, a surfactant (Tween 20 or Triton-X100), urea or guanidine, or an amino acid such as arginine or lysine can also be added, if necessary.

The salt for increasing the electrical conductivity is a salt comprising an alkali metal ion and/or an alkaline earth metal ion as a cation(s). In this step, the electrical conductivity of the eluent is increased in a continuous or stepwise manner based on the concentration change of the salt comprising an alkali metal ion and/or an alkaline earth metal ion. The alkali metal ion is not limited, but may be, for example, a lithium ion, a sodium ion, a potassium ion, or the like, or may be a sodium ion or a potassium ion. In other words, the salt for increasing the electrical conductivity may be, for example, a lithium salt, a sodium salt, a potassium salt, or the like, or may be a sodium salt or a potassium salt. The alkaline earth metal ion is not limited, but is, for example, a magnesium ion or a calcium ion. In other words, the salt for increasing the electrical conductivity is, for example, a magnesium salt or a calcium salt.

An anion comprised in the salt comprising an alkali metal ion and/or an alkaline earth metal ion is not particularly limited, but is, for example, a halide ion or a sulfate ion. The halide ion is, for example, a fluoride ion, a chloride ion, a bromide ion, or an iodide ion. In other words, the salt comprising an alkali metal ion and/or an alkaline earth metal ion is, for example, a halide or a sulfate. The halide is, for example, a fluoride, a chloride, a bromide, or an iodide. Specific examples of the halide include sodium chloride, potassium chloride, potassium iodide, calcium chloride, potassium bromide, sodium iodide, sodium bromide, and magnesium chloride. Additionally, specific examples of the sulfate include magnesium sulfate.

An elution rate in a case where a container such as a column packed with the carrier is used is not limited as long as the elution rate is a rate at which the eluent gently passes through the column, and may be, for example, a linear flow rate of 30 cm/h to 2000 cm/h. The amount of the eluent to be used may be 1 to 10 times the column volume, or 3 to 5 times the column volume.

In this step, the electrical conductivity of the eluent can be selected according to the size of a linear nucleic acid to be eluted. For example, an eluent of 10 mS/cm to 80 mS/cm, 20 mS/cm to 70 mS/cm, 30 mS/cm to 60 mS/cm, or 40 mS/cm to 50 mS/cm can be applied to the carrier to elute a linear nucleic acid of 1 kbp to 5 kbp or 2 kbp to 4 kbp. Additionally, an eluent of 1 mS/cm to 70 mS/cm, 10 mS/cm to 60 mS/cm, 20 mS/cm to 50 mS/cm, or 30 mS/cm to 40 mS/cm can be applied to the carrier to elute a linear nucleic acid of 0.5 kbp to 3 kbp or 1 kbp to 2 kbp. Furthermore, an eluent of 20 mS/cm to 90 mS/cm, 30 mS/cm to 80 mS/cm, 40 mS/cm to 70 mS/cm, or 50 mS/cm to 60 mS/cm can be applied to the carrier to elute a linear nucleic acid of 3 kbp to 7 kbp or 4 kbp to 6 kbp.

In the present specification, “increasing the electrical conductivity in a continuous manner” specifically means using an electrical conductivity gradient of the eluent. A method for adjusting the electrical conductivity gradient is well known in the art. The electrical conductivity gradient can be adjusted, for example, by mixing two or more buffer liquids having different electrical conductivities (for example, two buffer liquids differing only in sodium chloride concentration, such as a buffer liquid of 0.1 mS/cm and a buffer liquid of 100 mS/cm) while changing the mixing ratio in a continuous manner. In the present specification, “increasing the electrical conductivity in a stepwise manner” means using a plurality of eluents having different electrical conductivities. For example, a plurality of electrical conductivities may be used within the above-described range of the electrical conductivity.

The range of the electrical conductivity of the eluent applied to the carrier while increasing the electrical conductivity of the eluent in a continuous or stepwise manner is a range encompassing at least part or all of the range of 0.1 mS/cm to 150 mS/cm. For example, the electrical conductivity which is the starting point in the elution step is 0.1 mS/cm or higher, and may be 0.5 mS/cm or higher, 1 mS/cm or higher, 2 mS/cm or higher, 5 mS/cm or higher, 10 mS/cm or higher, 20 mS/cm or higher, or 30 mS/cm or higher. An exemplary range of the electrical conductivity which is the starting point is 0.1 mS/cm to 20 mS/cm or 1 mS/cm to 10 mS/cm. Additionally, the electrical conductivity which is the end point in the elution step is 150 mS/cm or lower, and may be, for example, 140 mS/cm or lower, 130 mS/cm or lower, 120 mS/cm or lower, 110 mS/cm or lower, 100 mS/cm or lower, 90 mS/cm or lower, 80 mS/cm or lower, 70 mS/cm or lower, or 60 mS/cm or lower. An exemplary range of the electrical conductivity which is the end point is 60 mS/cm to 150 mS/cm or 70 mS/cm to 100 mS/cm. By using the electrical conductivity in the above range, part or all of the linear nucleic acid can be dissociated from the carrier.

### (Collection Step)

In the separation method of one or more embodiments of the present invention, the “collection step” is a step of collecting the eluate. A specific collection method is not particularly limited as long as an eluate comprising a nucleic acid dissociated from the carrier can be collected. For example, when the separation method of one or more embodiments of the present invention uses a column packed with the carrier, the eluate eluted from the column may be collected by being divided into a plurality of fractions as necessary, and may be fractionated using, for example, a fraction collector. Additionally, in one or more embodiments in which the separation method of one or more embodiments of the present invention uses a mixture of a carrier such as a bead or a gel and the liquid sample, the collection step may be any method as long as the nucleic acid dissociated in the eluate can be separated from the carrier; depending on the type of carrier to be used, the carrier may be removed by, for example, sedimentation, aggregation, precipitation, filtration, centrifugation, or magnetic force, and the eluate comprising the nucleic acid eluted from the carrier may be collected.

Since the above-described elution step applies the eluent while increasing the electrical conductivity of the eluent in a continuous or stepwise manner, this step may collect the eluates eluted at different electrical conductivities, in the form of different fractions.

### (Purification Step)

In the separation method of one or more embodiments of the present invention, the “purification step” is a step of further purifying the eluate collected in the collection step. This step is an optional step.

A specific purification method is not limited, and examples thereof include: a separation method using gel extraction, cation exchange chromatography, anion exchange chromatography, size exclusion chromatography, a filter, or an ultrafiltration membrane; and dialysis. Purification may be performed using a commercially available nucleic acid purification kit. Other examples include a method of excising a range corresponding to a target size after electrophoresis using an acrylamide gel or an agarose gel.

### 1-4. Effects

According to the separation method of one or more embodiments of the present invention, linear nucleic acids such as two or more linear covalently closed DNAs can be efficiently separated based on a difference in molecular weight.

In the separation method disclosed in the above-mentioned Non-Patent Literature 1, a buffer liquid having a pH of 8.1 is used as an eluent for anion exchange membrane chromatography. The Literature discloses that pH 9.0, which is a pH higher than pH 8.1, can be an ideal condition in order to improve the collection rate. However, the below-described Examples of the present specification have revealed that linear covalently closed DNAs can be efficiently separated in a case where a buffer liquid of pH 4.0 to pH 6.5 is used as an eluent as compared with a case where a buffer liquid of pH 7.0 to pH 8.0 is used as an eluent. A possible reason for this is that the difference in the charge state of phosphate groups comprised in nucleic acid strands increases under a low pH condition.

In the separation method of one or more embodiments of the present invention, linear covalently closed DNAs can be efficiently separated particularly by using a bead having a particle diameter of 1 μm to 30 μm or 2 μm to 20 μm, for example, 1 μm to 10 μm.

### 2. Method for Producing Purified Nucleic Acid Product

### 2-1. Overview

One or more embodiments of the present invention are a method for producing a purified nucleic acid product (hereinafter referred to as a “method for producing a purified nucleic acid product” or simply “the production method of one or more embodiments of the present invention”). The production method of one or more embodiments of the present invention comprises a binding step, an elution step, and a collection step, and is characterized in that the elution of the nucleic acid in the elution step dissociates the purified nucleic acid product from the anion exchange group using an eluent of pH 4.0 to pH 6.5. According to the production method of one or more embodiments, a purified nucleic acid product comprising all or part of two or more linear nucleic acids in a different ratio can be produced from a liquid sample comprising the two or more linear nucleic acids such as linear covalently closed DNAs.

In the present specification, the “purified nucleic acid product” refers to a purified product comprising nucleic acids, obtained from a sample comprising at least two kinds of nucleic acids. In one or more embodiments, a liquid sample containing two or more linear nucleic acids having different molecular weights is subjected to the production method of one or more embodiments, and all or part of the two or more linear nucleic acids may be comprised in the purified nucleic acid product in a ratio different from the content ratio of the two or more linear nucleic acids in this liquid sample. For example, the abundance ratio of a target linear nucleic acid comprised in the two or more linear nucleic acids relative to other linear nucleic acids may increase by 5% or more, 10% or more, 20% or more, 30% or more, or 40% or more, 50% or more, 60% or more, 70% or more, 80% or more, 90% or more, or 100% or more. For example, 60% or more, 70% or more, 80% or more, 85% or more, 90% or more, 95% or more, 98% or more, 99% or more, or 99.5% or more of the nucleic acids comprised in the purified nucleic acid product may be linear nucleic acids having the same molecular weight, or substantially all of the nucleic acids may be linear nucleic acids having the same molecular weight.

### 2-2. Method

The production method of one or more embodiments of the present invention comprises a binding step, an elution step, and a collection step as essential steps, and comprises an equilibration step, a washing step, a purification step, and a preparation step as optional steps. The constitution of each step will be described below.

### (Equilibration Step)

In the production method of one or more embodiments of the present invention, the “equilibration step” is an optional step of equilibrating a carrier with a buffer liquid. Since this step conforms to the constitution of the equilibration step described above, the description thereof is omitted here.

### (Binding Step)

In the production method of one or more embodiments of the present invention, the “binding step” is a step of applying a liquid sample containing two or more linear nucleic acids having different molecular weights to a carrier having an immobilized anion exchange group to bind the two or more linear nucleic acids to the anion exchange group. Since this step conforms to the constitution of the binding step described above, a detailed description thereof is omitted here.

### (Elution Step)

In the production method of one or more embodiments of the present invention, the “elution step” is a step of applying an eluent of pH 4.0 to pH 6.5 to the carrier to elute a purified nucleic acid product dissociated from the anion exchange group. Since the specific constitution of this step conforms to the elution step described above, a detailed description thereof is omitted here.

### (Collection Step)

In the production method of one or more embodiments of the present invention, the “collection step” is a step of collecting an eluate comprising the purified nucleic acid product. Since the specific constitution of this step conforms to the collection step described above, a detailed description thereof is omitted here.

### (Preparation Step)

In the production method of one or more embodiments of the present invention, the “preparation step” is a step of preparing a nucleic acid solution comprising the purified nucleic acid product in a new buffer liquid after the collection step.

In this step, the “new buffer liquid” is not limited as long as the liquid is a buffer liquid having a composition different from the composition of the eluate collected in the above-described collection step. For example, the liquid may be a buffer liquid used for preserving nucleic acids in the art (for example, Tris-EDTA (TE) buffer liquid).

A specific preparation method used in this step is not limited. The buffer liquid can be replaced by, for example, sedimentation, aggregation, precipitation, centrifugation, filtration, and/or dialysis. Specifically, buffer exchange may be performed using gel extraction, cation exchange chromatography, anion exchange chromatography, size exclusion chromatography, a filter, or an ultrafiltration membrane. Purification may be performed using a commercially available kit. Note that a dry powder can also be prepared by further performing freeze-drying or the like on the nucleic acid solution prepared in this step.

### 3. Separation/Purification Kit

One or more embodiments of the present invention are a kit for separating or purifying linear nucleic acids (hereinafter referred to as a “separation/purification kit” or simply “the kit of one or more embodiments of the present invention”).

The kit of one or more embodiments of the present invention comprises, as essential components, a carrier to which an anion exchange group is immobilized, and an eluent of pH 4.0 to pH 6.5 for eluting nucleic acids. The eluent for eluting nucleic acids comprises a salt comprising an alkali metal ion and/or an alkaline earth metal ion.

Since the constitutions of the anion exchange group and the carrier conform to the description above, the description thereof is omitted here.

Additionally, the eluent for eluting nucleic acids also conforms to the constitution of the eluent used in the elution step described above. However, the upper limit of the electrical conductivity of the eluent for eluting nucleic acids comprised in the kit of one or more embodiments of the present invention is not limited. This is because the electrical conductivity can be adjusted by mixing the eluent with an eluent having a low electrical conductivity. The kit of one or more embodiments of the present invention may comprise two or more eluents having different electrical conductivities as the eluent for eluting nucleic acids, and may comprise, for example, two, three, four, five, or six or more eluents having different electrical conductivities.

The kit of one or more embodiments of the present invention may comprise, as an optional component, a wash liquid for washing the carrier to which the linear nucleic acid is bound. Additionally, a container for collecting the nucleic acid after elution, and/or instructions describing a method for separating the nucleic acid using the kit of one or more embodiments of the present invention may be comprised.

### EXAMPLES

One or more embodiments of the present invention will be specifically described below with reference to Examples. These Examples are for explaining one or more embodiments of the present invention, and do not limit the scope of the present invention.

### Example 1: Separation of Linear Covalently Closed DNA by Anion Exchange Column Chromatography

### (Purpose)

The purpose is to easily and efficiently separate large amounts of respective linear covalently closed DNAs from a liquid sample containing two types of linear covalently closed DNAs having different sizes, and to examine an equilibration buffer liquid and an eluent for eluting nucleic acids, in which the liquid and the eluent are used in anion exchange column chromatography.

### (Method)

### (1) Liquid DNA Mixture Sample

A liquid DNA mixture sample was prepared comprising: an approximately 9 kb plasmid DNA comprising a pair of protelomerase recognition sequences; an approximately 3 kb linear covalently closed DNA obtained by cleaving this plasmid DNA with a protelomerase; and an approximately 6 kb linear covalently closed DNA. In the following anion exchange column chromatography, 100 μL of a 150 ng/μL DNA mixture sample was loaded into a column (loading amount: 15 μg DNA).

### (2) Anion Exchange Column Chromatography

As an anion exchange column, Mini Q (trademark) 4.6/50 PE column (model number: 17517701, manufactured by Global Life Sciences Technologies Japan K.K.) was used. This anion exchange column is a product to be used with a column size of 0.8 mL (4.6 mm×50 mm), an injection amount of 15 μg, and a flow rate of 0.2 mL/min. The carrier constituting the column is a non-porous hydrophilic polymer (non-porous monodisperse polystyrene/divinyl benzene beads) (particle diameter 3 μm), and the charged group is —O—CH₂—CHOH—CH₂—O—CH₂—CHOH—CH₂—N⁺(CH₃)₃. The ion exchange capacity is 85 to 115 μmol Cl⁻/mL gel.

### (3) Compositions of Equilibration Buffer Liquid and Eluent for Eluting Nucleic Acids

For the equilibration buffer liquid, each composition described in Experimental Conditions 1 to 6 and Control Conditions 1 and 2 shown in Table 1 below was used. As the composition of the eluent for eluting nucleic acids, a composition obtained by adding 1 M NaCl to the composition of the equilibration buffer liquid under each condition was used.

TABLE 1
Composition of equilibration buffer liquid and composition of eluent for eluting nucleic acids

| | Composition of equilibration buffer | Composition of eluent for eluting nucleic |
|---|---|---|
| | liquid and electrical conductivity | acids and electrical conductivity |
| Experimental | 20 mM Bis-Tris-HCl, 1 mM EDTA, pH 6.5 | Composition on the left + 1M NaCl |
| Condition 1 | (electrical conductivity: 1.1 mS/cm) | (electrical conductivity: 78.9 mS/cm) |
| Experimental | 20 mM NaH₂PO₄—Na₂HPO₄, 1 mM EDTA, | Composition on the left + 1M NaCl |
| Condition 2 | pH 6.5 | (electrical conductivity: 74.2 mS/cm) |
| | (electrical conductivity: 2.1 mS/cm) | |
| Experimental | 20 mM histidine-HCl, 1 mM EDTA, pH 6.0 | Composition on the left + 1M NaCl |
| Condition 3 | (electrical conductivity: 1.2 mS/cm) | (electrical conductivity: 79.7 mS/cm) |
| Experimental | 20 mM citrate-NaOH, 1 mM EDTA, pH 5.0 | Composition on the left + 1M NaCl |
| Condition 4 | (electrical conductivity: 2.9 mS/cm) | (electrical conductivity: 79.8 mS/cm) |
| Experimental | 20 mM acetate-NaOH, 1 mM EDTA, pH 5.0 | Composition on the left + 1M NaCl |
| Condition 5 | (electrical conductivity: 1.2 mS/cm) | (electrical conductivity: 78.6 mS/cm) |
| Experimental | 20 mM acetate-NaOH, 1 mM EDTA, pH 4.0 | Composition on the left + 1M NaCl |
| Condition 6 | (electrical conductivity: 0.45 mS/cm) | (electrical conductivity: 79.0 mS/cm) |
| Control | 20 mM NaH₂PO₄—Na₂HPO₄, 1 mM EDTA, | Composition on the left + 1M NaCl |
| Condition 1 | pH 7.0 | (electrical conductivity: 80.4 mS/cm) |
| | (electrical conductivity: 2.6 mS/cm) | |
| Control | 20 mM Tris-HCl, 1 mM EDTA, pH 8.0 | Composition on the left + 1M NaCl |
| Condition 2 | (electrical conductivity: 1.3 mS/cm) | (electrical conductivity: 79.9 mS/cm) |

### (4) Program for Anion Exchange Column Chromatography

For each of the above conditions, chromatography was performed in accordance with the following steps (a) to (d):
(a) Column equilibration with equilibration buffer liquid (flow rate, 0.8 mL/min; linear flow rate, 289 cm/h)
(b) Loading of DNA mixture sample (flow rate, 0.2 mL/min; linear flow rate, 72 cm/h)
(c) Column washing with equilibration buffer liquid (flow rate, 0.2 mL/min; linear flow rate, 72 cm/h)
(d) Gradient elution from 0.7 M NaCl to 0.8 M NaCl (flow rate, 0.2 mL/min).

In step (d), gradient elution was performed by changing the mixing ratio of the eluent for eluting nucleic acids to the equilibration buffer liquid from 70:30 to 80:20 to change the electrical conductivity of the eluent in a continuous manner. The total amount of the eluate in step (d) was 16 mL, which was fractionated into 80 fractions of 0.2 mL each.

(5) Evaluation of Eluate from Anion Exchange Column Chromatography

The fractionated DNA was evaluated by an electrophoresis method. Specifically, 5 μL of the DNA solution after each elution was mixed with 1 μL of Gel Loading Dye, Purple (model number: B7025S, manufactured by New England Biolabs, Inc.). The resultant mixture was applied to a 1% agarose gel comprising 0.01% GelGreen (model number: 41004, manufactured by Biotium, Inc.), and analyzed by performing electrophoresis at 100 V for 40 minutes.

### (Results)

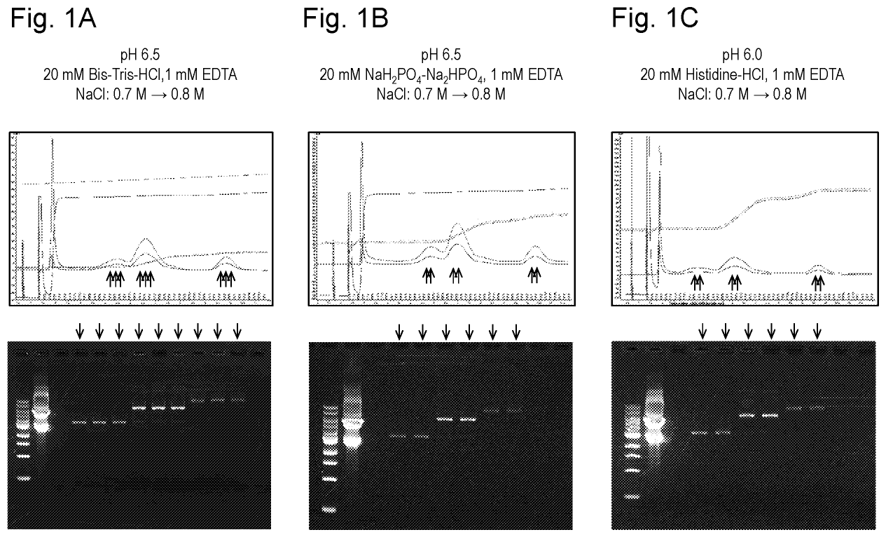

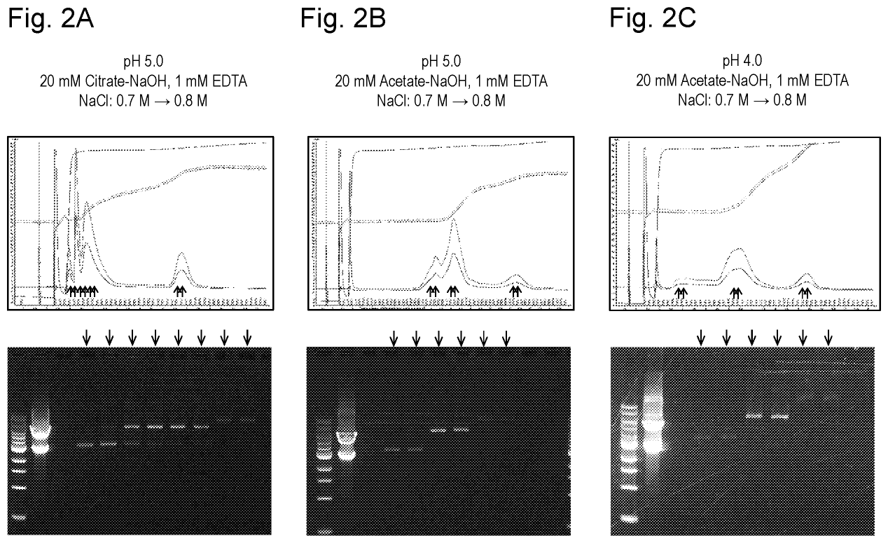

FIGS. 1A-1C and 2A-2C show the electrophoresis results of the DNA obtained by elution and fractionation under Experimental Conditions 1 to 6. In each panel of FIGS. 1A-1C and 2A-2C, the fractions eluted at the positions indicated by the arrows in the upper parts correspond to the lanes indicated by the arrows in the lower electrophoretogram. The results have revealed that the approximately 9 kb plasmid DNA, the approximately 3 kb linear covalently closed DNA, and the approximately 6 kb linear covalently closed DNA can be efficiently separated under Experimental Conditions 1 to 6 using the equilibration buffer liquid and the eluent for eluting nucleic acids in which the liquid and the eluent have a pH of 4.0 to 6.5.

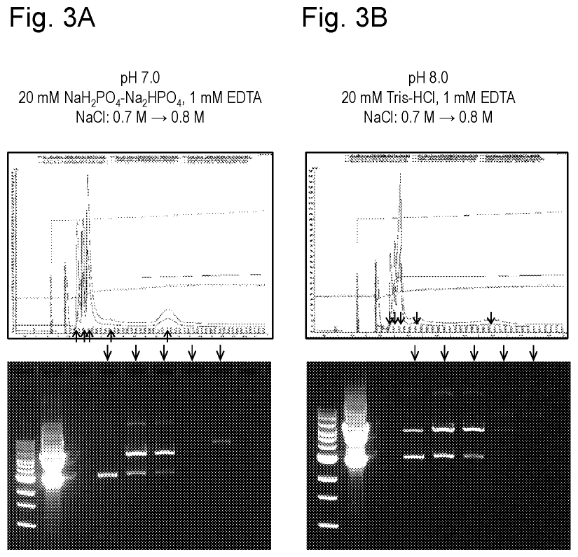

FIGS. 3A-3B show the electrophoresis results of the DNA obtained by elution and fractionation under Control Conditions 1 and 2. The results have revealed that the approximately 9 kb plasmid DNA, the approximately 3 kb linear covalently closed DNA, and the approximately 6 kb linear covalently closed DNA cannot be efficiently separated under Control Conditions 1 and 2 using the equilibration buffer liquid and the eluent for eluting nucleic acids in which the liquid and the eluent have a pH of 7.0 to 8.0.

### Example 2: Evaluation of Various Salts for Increasing Electrical Conductivity

### (Purpose)

The purpose is to evaluate effects based on various salts by performing anion exchange column chromatography using the various salts to increase the electrical conductivity. Specifically, the purpose is to verify that similar effects can be obtained by replacing the salt type used for increasing the electrical conductivity in Experimental Condition 3 of Example 1 from sodium chloride to eight types of salts (potassium chloride, potassium iodide, magnesium sulfate, calcium chloride, potassium bromide, sodium iodide, sodium bromide, and magnesium chloride).

### (Method)

### (1) Liquid DNA Mixture Sample

A DNA mixture liquid sample was prepared comprising an approximately 3 kb linear covalently closed DNA, an approximately 4.5 kb linear covalently closed DNA, and an approximately 9 kb linear covalently closed DNA. In the following anion exchange column chromatography, 100 μL of a 150 ng/μL DNA mixture sample was loaded into a column (loading amount: 15 μg DNA).

### (2) Anion Exchange Column Chromatography

As an anion exchange column, Mini Q (trademark) 4.6/50 PE column (model number: 17517701, manufactured by Global Life Sciences Technologies Japan K.K.) was used. This anion exchange column is a product to be used with a column size of 0.8 mL (4.6 mm×50 mm), an injection amount of 15 μg, and a flow rate of 0.2 mL/min. The carrier constituting the column is a non-porous hydrophilic polymer (non-porous monodisperse polystyrene/divinyl benzene beads) (particle diameter 3 μm), and the charged group is —O—CH₂—CHOH—CH₂—O—CH₂—CHOH—CH₂—N⁺ (CH₃)₃. The ion exchange capacity is 85 to 115 μmol Cl⁻/mL gel.

### (3) Compositions of Equilibration Buffer Liquid and Eluent for Eluting Nucleic Acids

As the equilibration buffer liquid, each composition described in Experimental Conditions (i) to (ix) shown in Table 2 below was used. As the composition of the eluent for eluting nucleic acids, a composition obtained by adding various salts (sodium chloride, potassium chloride, potassium iodide, magnesium sulfate, calcium chloride, potassium bromide, sodium iodide, sodium bromide, or magnesium chloride) to the composition of the equilibration buffer liquid under each condition was used.

TABLE 2
Composition of equilibration buffer liquid and composition of eluent for eluting nucleic acids

| | Composition of equilibration buffer | Composition of eluent for eluting nucleic |
|---|---|---|
| | liquid and electrical conductivity | acids and electrical conductivity |
| Experimental | 20 mM histidine-HCl, 1 mM EDTA, pH 6.0 | Composition on the left + 1M NaCl |
| Condition (i) | (electrical conductivity: 1.0 mS/cm) | (electrical conductivity: 82.5 mS/cm) |
| Experimental | 20 mM histidine-HCl, 1 mM EDTA, pH 6.0 | Composition on the left + 1M KCl |
| Condition (ii) | (electrical conductivity: 1.0 mS/cm) | (electrical conductivity: 103.8 mS/cm) |
| Experimental | 20 mM histidine-HCl, 1 mM EDTA, pH 6.0 | Composition on the left + 1M KI |
| Condition (iii) | (electrical conductivity: 1.0 mS/cm) | (electrical conductivity: 112.0 mS/cm) |
| Experimental | 20 mM histidine-HCl, 1 mM EDTA, pH 6.0 | Composition on the left + 2M MgSO₄ |
| Condition (iv) | (electrical conductivity: 1.0 mS/cm) | (electrical conductivity: 55.0 mS/cm) |
| Experimental | 20 mM histidine-HCl, 1 mM EDTA, pH 6.0 | Composition on the left + 1M CaCl₂ |
| Condition (v) | (electrical conductivity: 1.0 mS/cm) | (electrical conductivity: 122.6 mS/cm) |
| Experimental | 20 mM histidine-HCl, 1 mM EDTA, pH 6.0 | Composition on the left + 1M KBr |
| Condition (vi) | (electrical conductivity: 1.0 mS/cm) | (electrical conductivity: 108.4 mS/cm) |
| Experimental | 20 mM histidine-HCl, 1 mM EDTA, pH 6.0 | Composition on the left + 1M NaI |
| Condition (vii) | (electrical conductivity: 1.0 mS/cm) | (electrical conductivity: 85.5 mS/cm) |
| Experimental | 20 mM histidine-HCl, 1 mM EDTA, pH 6.0 | Composition on the left + 1M NaBr |
| Condition (viii) | (electrical conductivity: 1.0 mS/cm) | (electrical conductivity: 83.2 mS/cm) |
| Experimental | 20 mM histidine-HCl, 1 mM EDTA, pH 6.0 | Composition on the left + 1M MgCl₂ |
| Condition (ix) | (electrical conductivity: 1.0 mS/cm) | (electrical conductivity: 108.5 mS/cm) |

### (4) Program for Anion Exchange Column Chromatography

For each of the above conditions, chromatography was performed in accordance with the following steps (a) to (d):
(a) Column equilibration with equilibration buffer liquid (flow rate, 0.8 mL/min; linear flow rate, 289 cm/h)
(b) Loading of DNA mixture sample (flow rate, 0.2 mL/min; linear flow rate, 72 cm/h)
(c) Column washing with equilibration buffer liquid (flow rate, 0.2 mL/min; linear flow rate, 72 cm/h)
(d) Gradient elution (flow rate 0.2 mL/min).

In step (d), gradient elution was performed by changing the mixing ratio of the eluent for eluting nucleic acids and the equilibration buffer liquid to the ratio in Table 3 to change the electrical conductivity of the eluent in a continuous manner. The total amount of the eluate in step (d) is as shown in Table 3, and the eluate was fractionated into fractions of 0.2 mL each or 0.4 mL each.

TABLE 3
Mixing ratio of eluent for eluting nucleic acids to equilibration
buffer liquid in step (d), Total eluate volume

| | Change in mixing ratio of | Total |
|---|---|---|
| | eluent for eluting nucleic acids | eluate |
| | to equilibration buffer liquid | volume |
| Experimental Condition (i) | 70:30 to 80:20 | 16 mL |
| Experimental Condition (ii) | 75:25 to 80:20 | 24 mL |
| Experimental Condition (iii) | 30:70 to 45:55 | 24 mL |
| Experimental Condition (iv) | 30:70 to 45:55 | 24 mL |
| Experimental Condition (v) | 15:85 to 30:70 | 24 mL |
| Experimental Condition (vi) | 45:55 to 55:45 | 24 mL |
| Experimental Condition (vii) | 30:70 to 45:55 | 24 mL |
| Experimental Condition (viii) | 45:55 to 60:40 | 24 mL |
| Experimental Condition (ix) | 15:85 to 30:70 | 24 mL |

(5) Evaluation of Eluate from Anion Exchange Column Chromatography

The fractionated DNA was evaluated by an electrophoresis method. Specifically, 10 μL of the DNA solution after each elution was mixed with 2 μL of Gel Loading Dye, Purple (model number: B7025S, manufactured by New England Biolabs, Inc.). The resultant mixture was applied to a 1% agarose gel comprising 0.01% GelGreen (model number: 41004, manufactured by Biotium, Inc.), and analyzed by performing electrophoresis at 100 V for 40 minutes.

### (Results)

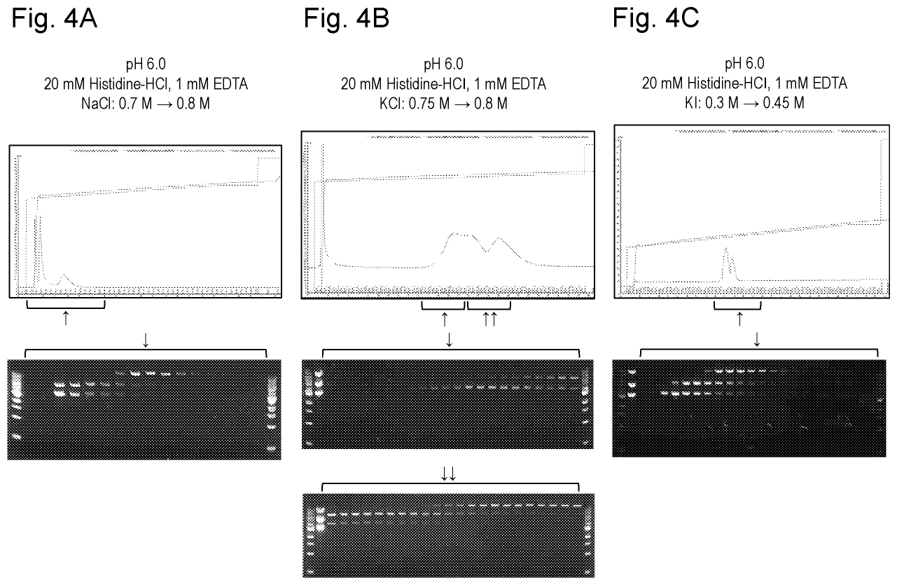

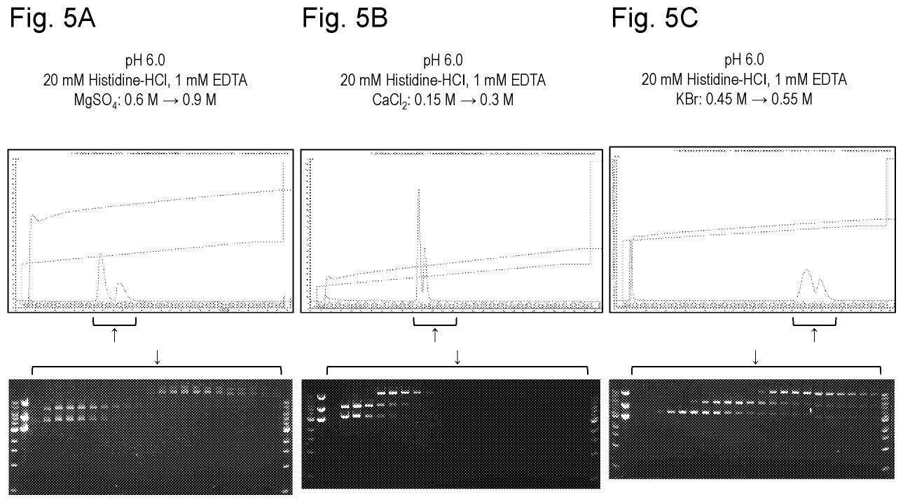

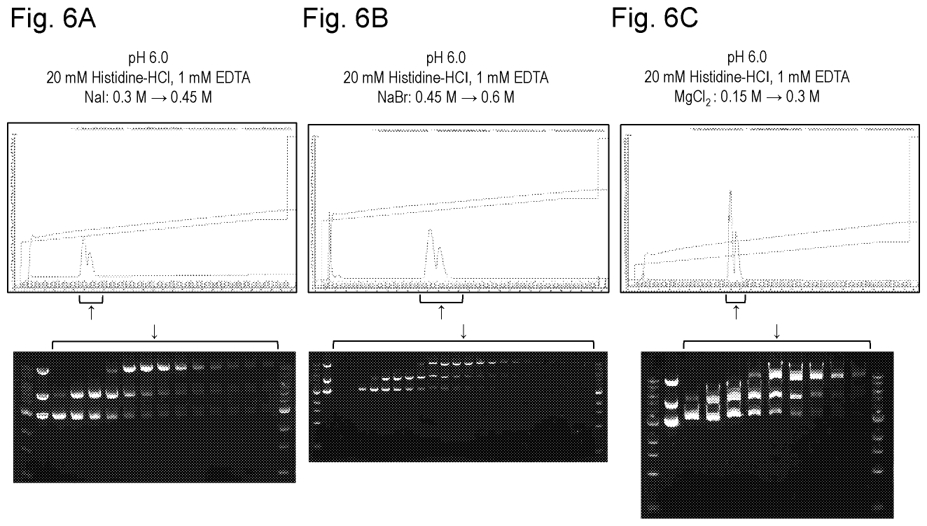

FIGS. 4A-4C, 5A-5C, and 6A-6C show the electrophoresis results of the DNA obtained by elution and fractionation under Experimental Conditions (i) to (ix). In each panel of the above Figures, the fractions eluted in the range in the chromatogram of the upper part correspond to the lanes in the lower electrophoretogram. The results have revealed that the approximately 3 kb linear covalently closed DNA, the approximately 4.5 kb linear covalently closed DNA, and the approximately 9 kb linear covalently closed DNA can be efficiently separated under Experimental Conditions (i) to (ix).

The above results have revealed that the use of the above eight kinds of salts (potassium chloride, potassium iodide, magnesium sulfate, calcium chloride, potassium bromide, sodium iodide, sodium bromide, and magnesium chloride) also enables efficient separation of two or more linear nucleic acids in the same manner as the use of sodium chloride.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

1. A method for separating two or more linear nucleic acids having different molecular weights, comprising:
applying a liquid sample containing the two or more linear nucleic acids to a carrier having an immobilized anion exchange group to bind the two or more linear nucleic acids to the anion exchange group;
applying an eluent of pH 4.0 to pH 6.5 to the carrier to elute an eluate containing a nucleic acid dissociated from the anion exchange group; and
collecting the eluate,
wherein the applying the eluent comprises increasing an electrical conductivity of the eluent in a continuous or stepwise manner based on a concentration change of a salt containing an alkali metal ion and/or an alkaline earth metal ion.

2. The method of claim 1, wherein the salt is a halide or a sulfate.

3. The method of claim 2, wherein the halide is selected from the group consisting of sodium chloride, potassium chloride, potassium iodide, calcium chloride, potassium bromide, sodium iodide, sodium bromide, and magnesium chloride.

4. The method of claim 2, wherein the sulfate is magnesium sulfate.

5. The method of claim 1, wherein a range for increasing the electrical conductivity encompasses at least part or all of the range of 0.1 mS/cm to 150 mS/cm.

6. The method of claim 1, wherein, in the collecting the eluate, the eluates eluted at different electrical conductivities are collected as different fractions.

7. The method of claim 1, wherein the eluent comprises a buffer liquid selected from the group consisting of Bis-Tris buffer liquid, phosphate buffer liquid, histidine buffer liquid, citrate buffer liquid, acetate buffer liquid, and combinations thereof.

8. The method of claim 1, wherein the anion exchange group is a strong anion exchange group.

9. The method of claim 8, wherein the strong anion exchange group comprises a quaternary amino group.

10. The method of claim 9, wherein the quaternary amino group is selected from the group consisting of a quaternary ammonium group, a quaternary aminoalkyl group, a quaternary aminoethyl group, a trimethylaminoethyl group, and combinations thereof.

11. The method of claim 1, wherein the two or more linear nucleic acids are DNA.

12. The method of claim 11, wherein the two or more linear nucleic acids are 2 kbp to 30 kbp.

13. The method of claim 1, wherein the two or more linear nucleic acids comprise a linear double-stranded DNA having one or both ends covalently closed.

14. A method for producing a purified nucleic acid product, comprising:
applying a liquid sample containing two or more linear nucleic acids having different molecular weights to a carrier having an immobilized anion exchange group to bind the two or more linear nucleic acids to the anion exchange group;
applying an eluent of pH 4.0 to pH 6.5 to the carrier to elute a purified nucleic acid product dissociated from the anion exchange group; and
collecting an eluate containing the purified nucleic acid product,
wherein the applying the eluent comprises increasing an electrical conductivity of the eluent in a continuous or stepwise manner based on a concentration change of a salt containing an alkali metal ion and/or an alkaline earth metal ion, and
wherein the purified nucleic acid product contains all or part of the two or more linear nucleic acids in a ratio different from the ratio in the liquid sample.

15. The method of claim 14, further comprising preparing a nucleic acid solution comprising the purified nucleic acid product in a buffer solution different from the eluate after the collecting the eluate.

16. A kit for separating or purifying linear nucleic acids, comprising:
a carrier to which an anion exchange group is immobilized; and
an eluent of pH 4.0 to pH 6.5 for eluting nucleic acids, comprising a salt containing an alkali metal ion and/or an alkaline earth metal ion.

17. The kit of claim 16, wherein an electrical conductivity of the eluent for eluting nucleic acids is 0.1 mS/cm or higher.

18. The kit of claim 16, wherein the anion exchange group is a strong anion exchange group.

19. The kit of claim 18, wherein the strong anion exchange group comprises a quaternary amino group.

20. The kit of claim 16, wherein the eluent for eluting nucleic acids comprises two or more eluents having different electrical conductivities.
